(19) United States
(12) Patent Application Publication
(10) Pub. No.: US 2026/0114924 A1
(43) Pub. Date: Apr. 30, 2026

(54) SKIN TREATMENT DEVICE

(71) Applicant: Shenzhen Ulike Smart Electronics Co., Ltd., Shenzhen (CN)

(72) Inventors: Lijun TAN, Shenzhen (CN); CHUANGYONG KE, Shenzhen (CN)

(21) Appl. No.: 19/163,528

(22) PCT Filed: Mar. 11, 2025
(86) PCT No.: PCT/CN2025/081711

§ 371 Date: Sep. 9, 2025

(30) Foreign Application Priority Data
Mar. 15, 2024 (CN) 202420521645.9
Jun. 19, 2024 (CN) 202421411754.1

(51) Int. Cl. A61B 18/18; A61B 18/00

(52) U.S. Cl. CPC A61B 18/18; A61B 2018/00005; A61B 2018/00077; A61B 2018/00095; A61B 2018/00178; A61B 2018/00476; A61B 2018/00642; A61B 2018/00773; A61B 2018/00904; A61B 2018/1807

(57) ABSTRACT

The present application provides a skin treatment device. The skin treatment device includes a light source assembly, a light-transmitting component, and a refrigeration component. The light-transmitting component has a light incident surface and a light output surface. The light incident surface faces the light source assembly, and the light incident surface can guide the light generated by the light source assembly into the light-transmitting component. The refrigeration component is located between the light-transmitting component and the light source assembly. The refrigeration component is in contact with the light incident surface, and at least a portion of the light incident surface is exposed from the refrigeration component.

### CROSS-REFERENCE TO RELATED APPLICATION(S)

The present application claims the benefit and priority to Chinese Patent Application Serial No. 202420521645.9, filed on Mar. 15, 2024, entitled “skin treatment device” and Chinese Patent Application Serial No. 202421411754.1, filed on Jun. 19, 2024, entitled “skin treatment device”, and the content of which is hereby fully incorporated by reference into the present application.

### FIELD

The subject matter relates to the technologies of beauty equipment, and more particularly, to a skin treatment device.

### BACKGROUND

Skin treatment devices, such as beauty devices including hair removal devices and photon rejuvenation devices, are used to treat the skin and achieve cosmetic effects. The hair removal device generally uses strong pulsed light to irradiate the skin. The light energy is absorbed by the hair follicles, which generates thermal energy to destroy the hair follicles, thereby achieving the therapeutic effect of hair removal. The photon rejuvenation device applies the strong pulsed light to the skin tissues to produce photothermal and photochemical effects, thereby rearranging the deep collagen fibers and elastic fibers to restore elasticity and improving the skin condition.

Due to the large thermal energy generated by the light, the light may cause burning sensation to the skin during work. In related arts, the skin treatment device generally uses sapphire as a light-transmitting component, which is in contact with the skin during work to avoid any burning sensation to the skin. In order to maintain the continuous cooling effect of the light-transmitting component on the skin, a cold end of a refrigeration component needs to be adhered to the light-transmitting component. In related arts, the skin treatment device usually arranges the refrigeration component at the side of the light-transmitting component to prevent a light incident surface from being blocked, resulting in an increase in the thickness of the light-transmitting component and an increase in cost. In addition, when the thickness of the light-transmitting component increases, there will be a greater loss of cooling capacity and a decrease in cooling efficiency due to the heat exchange of the light-transmitting component with the surrounding environment in the thickness direction.

### SUMMARY

In order to improve at least a portion of the shortcomings or deficiencies mentioned above, a skin treatment device is provided according to an embodiment of the present application.

Specifically, the skin treatment device provided by the embodiment of the present embodiment includes a light source assembly, a light-transmitting component, and a refrigeration component. The light source assembly includes a light incident surface and a light output surface. The light incident surface faces the light source assembly, and the light incident surface is configured to guide light generated by the light source assembly into the light-transmitting component. The refrigeration component is located between the light-transmitting component and the light source assembly. The refrigeration component is in contact with the light incident surface, and at least a portion of the light incident surface is exposed from the refrigeration component.

As can be seen from the above descriptions, the skin treatment device provided by the embodiment of the present application arranges the refrigeration component on the light incident surface of the light-transmitting component. Thus, the refrigeration component can cool the light-transmitting component through the light incident surface. Since there is no need to set the refrigeration component on the side of the light-transmitting component, the light-transmitting component can be made thinner, thereby reducing the manufacturing cost of the light-transmitting component. In addition, the surface area for heat exchange between the light-transmitting component and the air in the thickness direction is also reduced, thereby reducing the cooling capacity loss and improving the cooling efficiency.

### BRIEF DESCRIPTION OF THE DRAWINGS

Implementations of the present technology will now be described, by way of embodiment, with reference to the attached figures. Obviously, the drawings are only some embodiments of the present disclosure. For those ordinary skill in the art, other drawings can be obtained based on these drawings without creative work.

FIG. 1 is a structural schematic view of a skin treatment device according to an embodiment of the present application.

FIG. 2 is a structural schematic view of the skin treatment device in FIG. 1 after a housing is removed.

FIG. 3 is a structural schematic view showing a relationship between a light-transmitting component and a refrigeration component in FIG. 2.

FIG. 4 is a structural schematic view showing another relationship between the light-transmitting component and the refrigeration component in FIG. 2.

FIG. 5 is a structural schematic view of a relationship between the light-transmitting component, a thermal connector, the refrigeration component, and a support bracket in FIG. 2.

FIG. 6 is an exploded schematic view of the structure of FIG. 5.

FIG. 7 is an exploded schematic view of another skin treatment device according to an embodiment of the present application.

FIG. 8 is an exploded schematic view of the skin treatment device in FIG. 7.

FIG. 9 is an exploded schematic view of yet another skin treatment device according to an embodiment of the present application.

FIG. 10 is an exploded schematic view of the skin treatment device in FIG. 1.

FIG. 11 is a structural schematic view of a skin treatment device according to another embodiment of the present application.

FIG. 12 is a structural schematic view of a housing in FIG. 11.

FIG. 13 is a structural schematic view of a sensing medium in FIG. 11.

FIG. 14 is a cross-sectional view of the sensing medium in FIG. 13.

FIG. 15 is a structural schematic view of a skin sensing component in FIG. 11.

FIG. 16 is a structural schematic view showing the skin sensing component in FIG. 15 from another perspective.

FIG. 17 is a structural schematic view of a skin treatment device according to yet another embodiment of the present application.

FIG. 18 is an enlarged schematic view at VIII in FIG. 17.

FIG. 19 is a structural schematic view of a skin treatment device according to yet another embodiment of the present application.

FIG. 20 is an enlarged schematic view at X in FIG. 19.

FIG. 21 is an enlarged schematic view of a portion of the skin treatment device provided by yet another embodiment of the present application.

FIG. 22 is a structural schematic view of a skin treatment device according to yet another embodiment of the present application.

FIG. 23 is an enlarged schematic view at XII in FIG. 22.

FIG. 24 is a structural schematic view of a skin treatment device according to yet another embodiment of the present application.

FIG. 25 is an enlarged schematic view at XIV in FIG. 24.

### DETAILED DESCRIPTION

Implementations of the present disclosure will now be described, by way of embodiments, with reference to the above figures. The embodiments are obviously a portion but not all of the embodiments of the present application. Based on the embodiments of the present application, other embodiments obtained by ordinary skill in the art without creative work will still fall within the scope of protection of the present application.

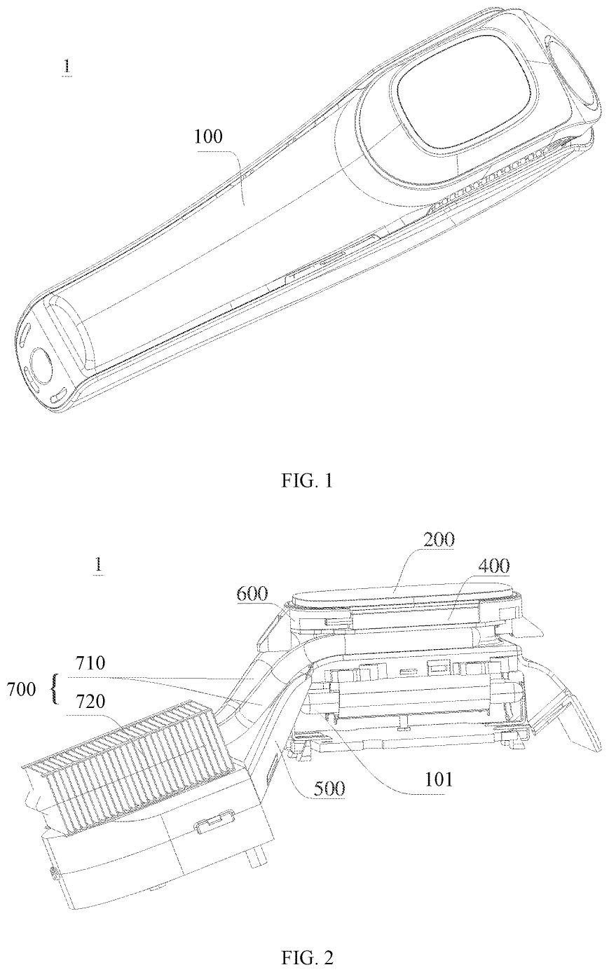

Referring to FIGS. 1 and 2, a skin treatment device 1 is provided according to an embodiment of the present application. The skin treatment device 1 may be a device such as a hair removal device or a photon rejuvenation device for treating the skin. In the embodiment of the present application, the skin treatment device 1 includes, for example, a light source assembly 101, a light-transmitting component 200, and a refrigeration component 400. Referring to FIG. 1, the skin treatment device 1 may further include a housing 100 for example for supporting and protecting the components in the skin treatment device 1.

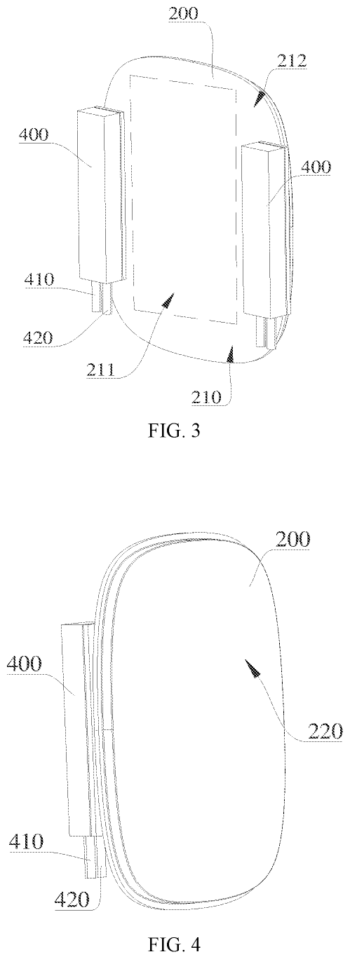

Specifically, referring to FIGS. 3 and 4, the light-transmitting component 200 has a light incident surface 210 and a light output surface 220 for example. The light incident surface 210 is arranged corresponding to the light source assembly 101, or the light incident surface 210 faces the light source assembly 101, and the light incident surface 210 is used to guide the light generated by the light source assembly 101 into the light-transmitting component 200. The refrigeration component 400 is located between the light-transmitting component 200 and the light source assembly 101 for example. The light source assembly 101 is used to generate the light, which may be an IPL lamp or a halogen lamp. The light-transmitting component 200 may be a sapphire for example, and is used to allow the light generated by the light source assembly 101 to pass through the light-transmitting component 200 and irradiate the skin. The refrigeration component 400, such as a semiconductor cooling chip, is used to cool the light-transmitting component 200 to reduce the burning and pain sensation generated by the skin treatment device 1 during the skin treatment. The refrigeration component 400 is in contact with the light incident surface 210, and at least a portion of the light incident surface 210 is exposed from the refrigeration component 400. In this embodiment, the refrigeration component 400 is provided at an edge portion of the light incident surface 210 for example. A center portion of the light incident surface 210 is used to transmit the light generated by the light source assembly 101 to ensure sufficient area of the light incident surface 210, such that more energy of the light can be transmitted from the light incident surface 210 to the light output surface 220, thereby improving the light output effect of the skin treatment device 1.

In this embodiment, taking the skin treatment device 1 as a hair removal device for example, when the hair removal device is used for hair removal, the light source assembly 101 is an IPL lamp for generating light beams for example, and the light beams will pass through the light-transmitting component 200 onto the skin. The refrigeration component 400 is located on the light incident surface 210 of the light-transmitting component 200 and in contact with the light incident surface 210. By setting a cold end of the refrigeration component 400 to be in contact with the light incident surface 210, the cooling capacity of the refrigeration component 400 will be transmitted to the light-transmitting component 200, thereby cooling the light-transmitting component 200 and reducing the high-temperature burning sensation caused by the light emission from the IPL lamp during the hair removal. In related arts, the refrigeration component 400 is installed on a side of the light-transmitting component 200, resulting in an increase in the thickness of the light-transmitting component 200, thereby increasing manufacturing costs. In addition, since the refrigeration component 400 is placed on the side of the light-transmitting component 200, the light-transmitting component 200 is usually made into a hexahedron (a block). Thus, except for the side of the light-transmitting component 200 that is in contact with the refrigeration component 400, the other surfaces not being in contact with the refrigeration component 400 will exchange heat with air or the housing 100, resulting in loss of the cooling capacity of the light-transmitting component 200. In order to solve the problem of poor light output and cooling effect in related arts, the thickness of the light-transmitting component 200 may generally be made thicker, that is, the thickness of the light-transmitting component 200 is increased. As the thickness of the light-transmitting component 200 increases, the surface area of the light-transmitting component 200 that does not come into contact with the refrigeration component 400 will also increase, resulting in a greater loss of cooling capacity after heat exchange. In the present application, by setting the refrigeration component 400 at the light incident surface 210 of the light-transmitting component 200, the thickness of the light-transmitting component 200 can be reduced, and the surface area through which the light-transmitting component 200 exchanges heat with air will be smaller, thereby reducing the loss of cooling capacity.

The skin treatment device 1 according to the embodiment of the present application sets the refrigeration component 400 at the light incident surface 210 of the light-transmitting component 200, such that the refrigeration component 400 can cool the light-transmitting component 200 through the light incident surface 210. Since the refrigeration component 400 is not required to be provided at the side of the light-transmitting component 200, the light-transmitting component 200 can be made thinner. Compared with the block-shaped light-transmitting component, the manufacturing cost of a sheet-shaped light-transmitting component is lower, thereby reducing the manufacturing cost of the light-transmitting component 200. In addition, the surface area through which the light-transmitting component 200 exchanges heat with the air in the thickness direction is also reduced, thereby reducing the loss of cooling capacity and improving the cooling efficiency.

In addition, in the case where the size of the light outlet of the skin treatment device 1 is constant, in order to ensure the irradiation intensity, the light incident area of the light-transmitting component should match the size of the light outlet as much as possible. In related arts where the refrigeration component is set on the side of the block-shaped light-transmitting component, the size of each of the light incident surface and the light output surface of the block-shaped light-transmitting component is substantially the same as the size of the light outlet. The surface area of the light incident surface is in fact the light incident area of the light-transmitting component. However, the embodiment of the present application, the light incident surface 210 of the sheet-shaped light-transmitting component 200 needs to leave a space to set the refrigeration component 400. Therefore, without sacrificing the light incident area, the size of the light incident surface 210 is set to be larger than the size of the light outlet. That is, the size of the sheet-shaped light-transmitting component 200 is set to be larger, and the light output area of the light-transmitting component 200 also increases accordingly. Thus, the area of the light-transmitting component 200 in contact with the skin also increases, thereby improving the cooling effect and light output efficiency, and enhancing skin treatment efficiency.

Referring to FIGS. 3 and 4, the light incident surface 210 includes a light incident area 211 and a contact area 212. The light incident area 211 is the area enclosed by the dashed lines shown in FIG. 3 for example. The light incident area 211 is located at the interior area such as the center area of the light incident surface 210, and the contact area 212 is located at the edge area of the light incident surface 210. In some embodiments, the contact area 212 surrounds the light incident area 211. In other embodiments, the distribution of the contact area 212 and the light incident area 211 may also be adjusted according to the actual design of the product, and is not limited. The light incident area 211 is used to guide the light generated by the light source assembly 101 into the light-transmitting component 200, and the refrigeration component 400 is in contact with the contact area 212.

In this embodiment, the light source assembly 101 generates the light, which enters the light-transmitting component 200 through the light incident area 211. The light incident area 211 is located at the center area of the light incident surface 210, which is conducive to accurately guiding light. The refrigeration component 400 is located at the contact area 212 and in contact with the contact area 212, thereby ensuring that the refrigeration component 400 effectively provides cooling capacity to the translucent component 200, and reducing the pain and burning sensation during the skin treatment. The refrigeration component 400 is set on the contact area 212 of the light-transmitting component 200 and in contact with the contact area 212. The refrigeration component 400 will not block the light incident area 211 of the light-transmitting component 200, such that the light output area when the light is generated by the light source assembly 101 and output from the light output surface 220 of the light-transmitting component 200 is not affected by the refrigeration component 400. Therefore, the cooling area and the light output surface 220 of the light-transmitting component 200 may be enlarged to improve the cooling effect and light output efficiency, and there is no need to increase the thickness of the light-transmitting component 200. In this embodiment, the light-transmitting component 200 is, for example, a sapphire crystal. The thinner the light-transmitting component 200, the more sapphire chips are cut therefrom. Thus, the thinner the light-transmitting component 200, the lower the cost of using the light-transmitting component 200. In other embodiments, the light-transmitting component 200 may also be a quartz glass or K9 glass for example. The light-transmitting component 200 may be any light-transmitting component that can reduce the burning sensation caused by the light emitted by the light source assembly 101 on the skin, and is not limited.

A distance from the light incident surface 210 to the light output surface 220 is 1 mm to 10 mm. By limiting the distance from the light incident surface 210 to the light output surface 220 in this embodiment, the light may pass through a short distance from the light incident surface 210 to the light output surface 220 of the light-transmitting component 200, thereby reducing energy loss and light diffusion, and improving light output efficiency and cooling effect. Thus, more light is allowed to accurately irradiate the skin to achieve better therapeutic effects. In one embodiment, the distance from the light incident surface 210 to the light output surface 220 is 1 mm. In another embodiment, the distance from the light incident surface 210 to the light output surface 220 may also be 5 mm. In yet another embodiment, the distance from the light incident surface 210 to the light output surface 220 may also be 10 mm. Optionally, the distance from the light incident surface 210 to the light output surface 220 is 1.5 mm to 5 mm in this embodiment. By limiting the distance from the light incident surface 210 to the light output surface 220, the converging degree of the light and the thermal conduction distance can be controlled, thereby controlling the heat transfer between the light-transmitting component 200 and the skin, avoiding excessive heat transfer to the skin to reduce pain sensation, and improving comfort and safety. Thus, the skin treatment device 1 has good light output and cooling effects while being cost-effective.

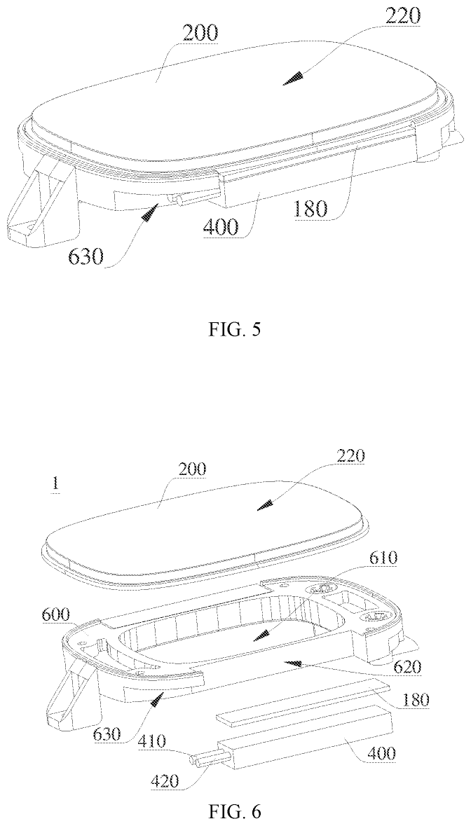

Referring to FIGS. 5 and 6, the skin treatment device 1 may further includes, for example, a support bracket 600. The support bracket 600 is set on a side of the light-transmitting component 200 closing to (facing) the light source assembly 101. The support bracket 600 is connected to the light-transmitting component 200 and in contact with the contact area 212. The support bracket 600 may be an independent component, and may also be a portion of the housing 100, a heat dissipation component, an installation bracket, or another component, which is limited in the present application.

The support bracket 600 defines a light incident hole 610. The light incident area 211 corresponds to the light incident hole 610. Therefore, the light generated by the light source assembly 101 can pass through the light incident hole 610 and the support bracket 600, further enter the light-transmitting component 200 through the light incident area 211 of the light incident surface 210, and finally output from the light output surface 220.

The support bracket 600 has an installation space 620, and the refrigeration component 400 is installed in the installation space 620. The installation space 620 may be located at the side of the support bracket 600 for example. The side of the support bracket 600 may be recessed inward to form the installation space 620 for example. The support bracket 600 provides additional support and protection functions, thereby avoiding external impacts or damages to components such as the light-transmitting component 200 and the refrigeration component 400 installed on, improving the stability and durability of the device, extending the service life, and achieving excellent cooling effect and structural stability of the skin treatment device 1.

Referring to FIG. 6, the installation space 620 is located adjacent to the light incident hole 610. The refrigeration component 400 is embedded in the installation space 620 and exposed from the surface of the support bracket 600. By setting the light incident hole 610 in this embodiment, the light may pass through the light incident hole 610 of the support bracket 600 and then enter the light-transmitting component 200, thereby achieving accurate light transmission. The refrigeration component 400 is embedded in the installation space 620 of the support bracket 600 and exposed from the surface of the support bracket 600. By exposing the refrigeration component 400 from the surface of the support bracket 600, the heat dissipation is enhanced to maintain the working temperature of the refrigeration component 400. As such, when the refrigeration component 400 is working, the light-transmitting component 200 is cooled more effectively, thereby optimizing the cooling structure, improving the cooling effect of the skin treatment device 1, reducing the temperature of the skin and reducing the pain sensation.

Furthermore, there are multiple refrigeration components 400 and multiple installation spaces 620. The refrigeration components 400 corresponds to the installation spaces 620 one-to-one. The installation spaces 620 are distributed on the side of the support bracket 600. The refrigeration components 400 surround the light incident hole 610 and is in contact with the contact area 212. By setting the refrigeration components 400 to correspond to the installation spaces 620 one-to-one, the refrigeration components 400 can cool the light-transmitting component 200 to improve the cooling effect of the light-transmitting component 200. When there are two installation spaces 620, the two installation spaces 620 may be set on the left and right sides of the support bracket 600 as shown in FIG. 8. When there are three installation spaces 620, two of the three installation spaces 620 are set on the left and right sides of the support bracket 600, and the remaining one of the three installation spaces 620 is set on the upper or lower side of the support bracket 600. The installation space 620 provides an installation location for the refrigeration component 400, such that the refrigeration component 400 can be fully in contact with the area that needs to be cooled, thereby achieving effective cooling effect. By disturbing the installation spaces 620 on the side of the support bracket 600, reasonable utilization of the space is achieved, thereby avoiding occupying additional installation locations, and improving the internal space utilization and installation flexibility of the device. In addition, since the refrigeration components 400 surround the light incident hole 610, the area near the light incident hole 610 can be cooled, thereby effectively reducing the temperature, and improving the working efficiency of the system in high temperature environments.

Referring to FIGS. 5 and 6, the skin treatment device 1 further defines a wire space 630. The wire space 630 may communicate with the installation space 620 for example, which allows the structure of the skin treatment device 1 to be more compact to reduce the volume. The refrigeration component 400 further includes a first wire 410 and a second wire 420. Each of the first wire 410 and the second wire 420 extends through the wire space 630. One of the first wire 410 and the second wire 420 is connected to a positive pole of the power supply, and the other of the first wire 410 and the second wire 420 is connected to the negative pole of the power supply. By connecting the wire space 630 to the installation space 620, electrical energy can be transmitted to the refrigeration component 400 through the first wire 410 and the second wire 420. In this embodiment, the first wire 410 and the second wire 420 are located at the same end of the refrigeration component 400, which is beneficial for reducing the winding distance and making the structure of the skin treatment device 1 more compact.

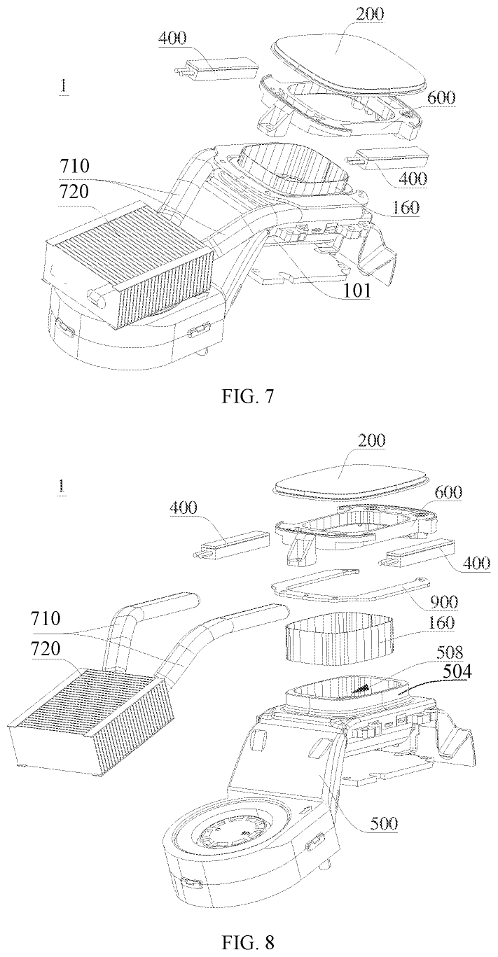

Referring to FIGS. 7 and 8, the skin treatment device 1 further includes an installation bracket 500 and a heat dissipation component 700. The installation bracket 500 is located at a side of the support bracket 600 closing (facing) the light source assembly 101. The light source assembly 101 is mounted on the installation bracket 500. The heat dissipation component 700 is positioned between the support bracket 600 and the installation bracket 500. The heat dissipation component 700 is connected to and thermally conductive with one end of the refrigeration component 400 away from the light incident surface 210. The heat dissipation component 700 includes, for example, a thermal conducting component 710 and a temperature equalizing component 720 as shown in FIGS. 7 and 8. The thermal conducting component 710 is, for example, a thermal conducting pipe, and the temperature equalizing component 720 is, for example, a temperature equalizing plate for heat dissipation. The thermal conducting pipe is used to absorb the heat generated at the hot end of the refrigeration component 400 and transfer the heat to the temperature equalization plate. In this embodiment, there may be multiple temperature equalization plates arranged at intervals, such that the heat transferred to the temperature equalization plates is dissipated in a divergent manner. By setting the installation bracket 500 at the side facing the light source assembly 101 and arranging the light source assembly 101 inside the installation bracket 500, the position of the light source assembly 101 is more stable, thereby reducing the displacement of the light or the damages to light source assembly 101 under external vibration or collision, and ensuring safe use. The heat dissipation component 700 is located between the support bracket 600 and the installation bracket 500 and connected to and thermally conductive with the hot end of the refrigeration component 400, thereby improving the heat dissipation effect of the refrigeration component 400 and ensuring the stability and service life of the skin treatment device 1.

Referring to FIG. 8, the skin treatment device 1 further includes a conductive component 900. The conductive component 900 is, for example, made of a thermally conductive material. The conductive component 900 is located between the support bracket 600 and the heat dissipation component 700. The conductive component 900 is connected to and thermally conductive with the refrigeration component 400 and the heat dissipation component 700. The conductive component 900 is, for example, U-shaped as shown in FIG. 8. In other embodiments, the conductive component 900 may also be, for example, a semi-annular or annular, which is not limited. The conductive component 900 is used to transfer the heat generated by the hot end of refrigeration component 400 to heat dissipation component 700 through contact heat dissipation. Since the conductive component 900 is connected to and thermally conductive with the refrigeration component 400 and the heat dissipation component 700, the heat dissipation area between the refrigeration component 400 and the heat dissipation component 700 is increased, thereby improving the heat conduction efficiency. The conductive component 900 with a semi-annular or annular shape can better adapt to the layout of the refrigeration component 400 and prevent the light incident area of the light incident surface 210 from being blocked.

Referring to FIG. 8, the installation bracket 500 defines a through hole 508 protruding toward the support bracket 600. That is, the installation bracket 500 is provided with a flange 504 protruding toward the support bracket 600, and the flange 504 defines the through hole 508 therein. The through hole 508 communicates with the light incident hole 610. The skin treatment device 1 may further include a light converging component 160 for example, which is used to converge the light generated by the light source assembly 101, thereby allowing more light to be emitted from the light output surface 220. The light converging component 160 is, for example, set on the inner wall of the light incident hole 610 or on the inner wall of the through hole 508. In other embodiments, a portion of the light converging component 160 may be set on the inner wall of the light incident hole 610, and another portion of the light converging component 160 may be set on the inner wall of the through hole 508, and the specific details are not limited. Since the through hole 508 communicates with the light incident hole 610, the light can be transmitted from the light source assembly 101 to the light incident hole 610 through the through hole 508. The through hole 508 and the light incident hole 610 are conductive to guide the light transmission and adjust the position of the light converging component 160 to meet different needs. In this embodiment, the light converging component 160 is, for example, a sheet-shaped structure that can focus the light.

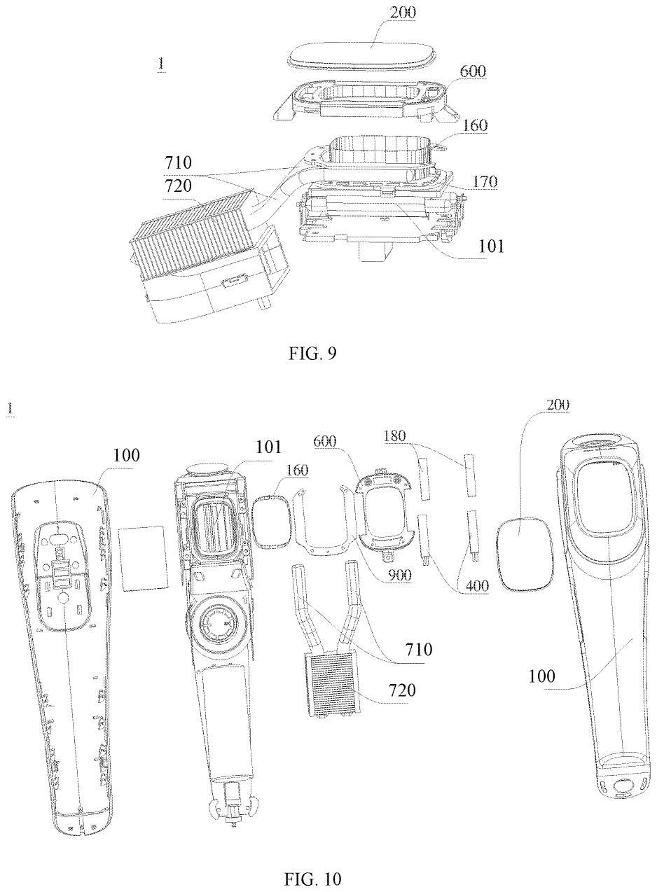

Referring to FIG. 9, the skin treatment device 1 further includes an auxiliary light emitting component 170. The auxiliary light emitting component 170 may be, for example, located between the light source assembly 101 and the light-transmitting component 200. When the skin treatment device 1 is working, the light generated by the light source assembly 101 first passes through the auxiliary light emitting component 170 and then output from the light-transmitting component 200. The auxiliary light emitting component 170 can achieve uniform light distribution by adjusting and evenly distributing the light, such that the light generated by the light source assembly 101 is evenly distributed on the light-transmitting component 200, avoiding local brightness being too high or too low, and providing a more uniform and consistent lighting effect. In addition, by setting the auxiliary light emitting component 170, the light source assembly 101 and the internal stacking structures can be shielded, thereby avoiding users from seeing the internal stacking structures through the light-transmitting component 200, increasing the aesthetic effect of the skin treatment device 1, and enhancing the user experience. In this embodiment, the auxiliary light emitting component 170 is, for example, a sheet-shaped structure that can diffuse the light.

Referring to FIGS. 5, 6, and 10, the skin treatment device 1 further includes, for example, a thermal conductive connector 180. The thermal conductive connector 180 is, for example, made of thermal conductive silicone grease. The thermal conductive connector 180 is located between the light-transmitting component 200 and the refrigeration component 400. The thermal conductive connector 180 is in contact with the light incident surface 210. The thermal conductive connector 180 is connected to and thermally conductive with the light-transmitting component 200 and the refrigeration component 400. When the light source assembly 101 generates heat, the thermal conductive connector 180 comes into contact with the light-transmitting component 200 and the refrigeration component 400, and quickly transfers the heat to the refrigeration component 400 through thermal conduction, thereby providing an efficient thermal conduction channel that allows the heat to be quickly transferred to the refrigeration component 400, thus effectively dissipating the heat. By setting the thermal conductive connector 180, the light-transmitting component 200 can maintain a uniform temperature distribution, thereby ensuring even output of light. The thermal conductive connector 180 effectively controls the temperature of the light source assembly 101, thereby preventing temperature fluctuations and temperature instability caused by overheating and maintaining stable operation of the device.

In related arts, the skin treatment device uses the housing as a sensing medium of a skin sensing component. The inventor discovered through creative labor that the sapphire of the skin treatment device can be designed with a larger skin contact area to increase the cosmetic efficiency of the skin treatment device. When the skin of the user only comes into contact with the sapphire of the skin treatment device, the skin treatment device fails to recognize the skin in contact with the sapphire through the skin sensing component at the housing, resulting in failures of the light source assembly of the skin treatment device to illuminate.

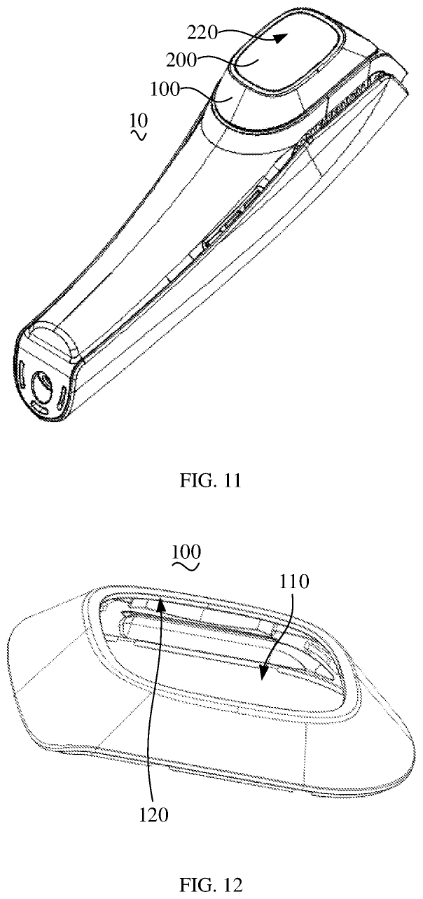

In view of the above description, a skin treatment device with a large-area sapphire for implanting an improved skin sensing solution is provided according to another embodiment of the present application. Referring to FIG. 11, the skin treatment device 10 of the embodiment of the present application may be a hair removal device. The hair removal device can emit a series of strong pulsed light. The light includes multiple wavelengths, which can be absorbed by melanin in hair follicles. After the hair follicles absorb the light energy, the temperature of the hair follicles rapidly increases, causing damage to the hair follicle structures, thereby inhibiting or delaying the hair regeneration. Alternatively, the skin treatment device 10 may also be a beauty device. The beauty device can stimulate the skin to produce biological reactions, such as promoting the regeneration of collagen and elastic fibers, thereby improving microcirculation and enhancing the self-repair ability of the skin. The reactions also help to improve skin texture, enhance firmness and radiance, and achieve a rejuvenating effect. The technical features of the skin treatment device 10 of the embodiment of the present application may be combined with the technical features of the skin treatment device 1 mentioned above as long as they do not conflict with each other.

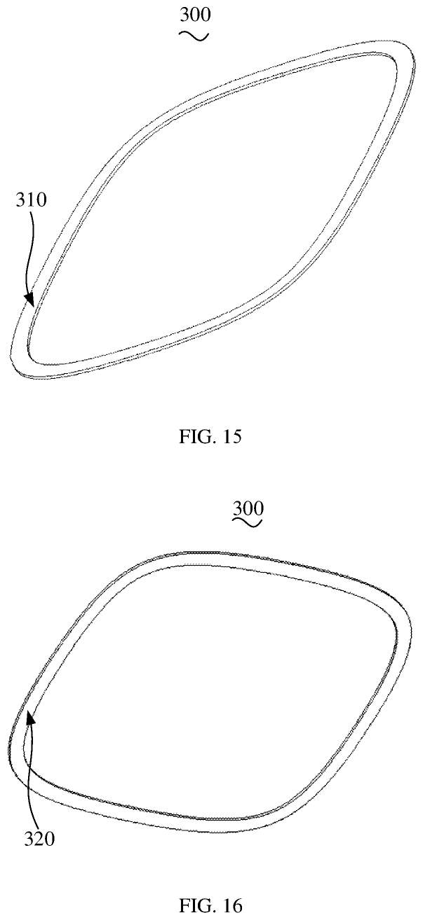

Referring to FIGS. 11 to 18, the skin treatment device 10 may include a housing 100, a sensing medium 200, and a skin sensing component 300. The housing 100 may be provided with a light source assembly 101, and the sensing medium 200 may be the light-transmitting component 200 of the skin treatment device 1 mentioned above. The sensing medium 200 is made of crystalline material. A portion of the sensing medium 200 is exposed from the surface of the housing 100 and used to output the light generated by the light source assembly 101. The skin sensing component 300 is at least partially stacked with the sensing medium 200. Compared to the skin treatment device 10 in related arts, the skin treatment device 10 of the present application stacks at least a portion of the skin sensing component 300 with the sensing medium 200 made of crystal material. When the skin of the user comes into contact with the sensing medium 200, the skin treatment device 10 can determine that the skin is in contact with the sensing medium 200 through the skin sensing component 300, such that the skin treatment device 10 can control the light from the light source assembly 101 to output.

In addition, when the skin is in contact with the sensing medium 200, the air gap between the skin surface and the sensing medium 200 can be reduced, which helps to reduce the risk of the light generated by the light source assembly 101 being reflected or scattered into the air after the light passes through the sensing medium 200, thereby allowing more light energy from the light source assembly 101 to reach the skin for treatment or care purposes. Moreover, since the sensing medium 200 is made of a crystalline material with high thermal conductivity, the sensing medium 200 can quickly absorb the heat from the skin, thereby allowing the sensing medium 200 to apply cold compress on the skin, and effectively reducing the surface temperature of the skin to reduce the risk of skin burnings during phototherapy.

It can be understood that the skin sensing component 300 may be in directly contact with the sensing medium 200, that is, the skin sensing component 300 is directly adhered to the sensing medium 200. Alternatively, the skin sensing component 300 may also be in indirectly contact with the sensing medium 200, that is, the skin sensing component 300 is connected to the sensing medium 200 through a conductor.

There are various choices for the sensing medium 200. For example, the sensing medium 200 may be sapphire. For example, the sensing medium 200 may also be Pato stone. For example, the sensing medium 200 may also be crystal, which can be selected according to actual situations.

There are various ways for the skin treatment device 10 to determine whether the skin is in contact with the sensing medium 200 through the skin sensing component 300. For example, the skin sensing component 300 may be a pressure sensor. When the skin is in contact with the sensing medium 200, the skin will slightly press the sensing medium 200, and the slight pressure generated by the skin that is in contact with the sensing medium 200 will be captured by the skin sensing component 300. The skin sensing component 300 can be equipped with a deformable element (such as a strain gauge) therein. When subjected to a pressure force, the deformable element will undergo small deformation after being compressed, resulting in a change in resistance value of the deformable element. Such change in resistance value can be converted into an electrical signal that is transmitted to a circuit board of the skin treatment device 10, such that the skin treatment device 10 can determine that the skin is in contact with the sensing medium 200, and thus the light source assembly 101 can be controlled to start to work or stop working.

For example, the skin sensing component 300 may also be a photoelectric sensor. The sensing medium 200 may define a light through hole. The skin sensing component 300 may include a transmitter and a receiver. The transmitter and the receiver may be located on a same side of the light through hole. The transmitter may emit light through the light through hole. When the skin is in contact with the sensing medium 200, the skin blocks the light through hole. At this time, the light emitted by the transmitter will be reflected by the skin and received by the receiver. After the receiver receives the light, the light may be converted into an electrical signal that is transmitted to the circuit board of the skin treatment device 10, such that the skin treatment device 10 can determine that the skin is in contact with the sensing medium 200, and thus the light source assembly 101 can be controlled to start to work or stop working.

For example, the skin sensing component 300 may also be a capacitive sensor. When the skin is not in contact with the sensing medium 200, the skin sensing component 300 serves as a reference capacitor, and at this time, a total capacitance value of the capacitance circuit of the skin sensing component 300 is the value of a reference capacitor. When the skin comes into contact with the sensing medium 200, the skin sensing component 300 serves as a reference capacitor, the skin serves as one pole plate of the capacitor, and the sensing medium 200 made of crystal material serves as an insulating medium of the capacitor. Thus, the skin, the sensing medium 200, and one pole plate of the skin sensing component 300 cooperatively form an identification capacitor. The identification capacitor is connected in parallel with the reference capacitor, and at this time, the total capacitance value of the capacitance circuits of the skin sensing component 300 is the sum of the value of the identification capacitor and the value of the reference capacitor. Furthermore, the skin sensing component 300 is electrically connected to the circuit board. The circuit board can determine whether the skin is in contact with the sensing medium 200 based on the total capacitance value of the capacitance circuits of the skin sensing component 300, such that the skin treatment device 10 can determine that the skin is in contact with the sensing medium 200, and thus the light source assembly 101 can be controlled to start to work or stop working.

The following embodiments are described when taking the skin sensing component 300 as the capacitive sensor for example. The embodiments of the skin sensing component 300 as other sensors can be referred to the following descriptions.

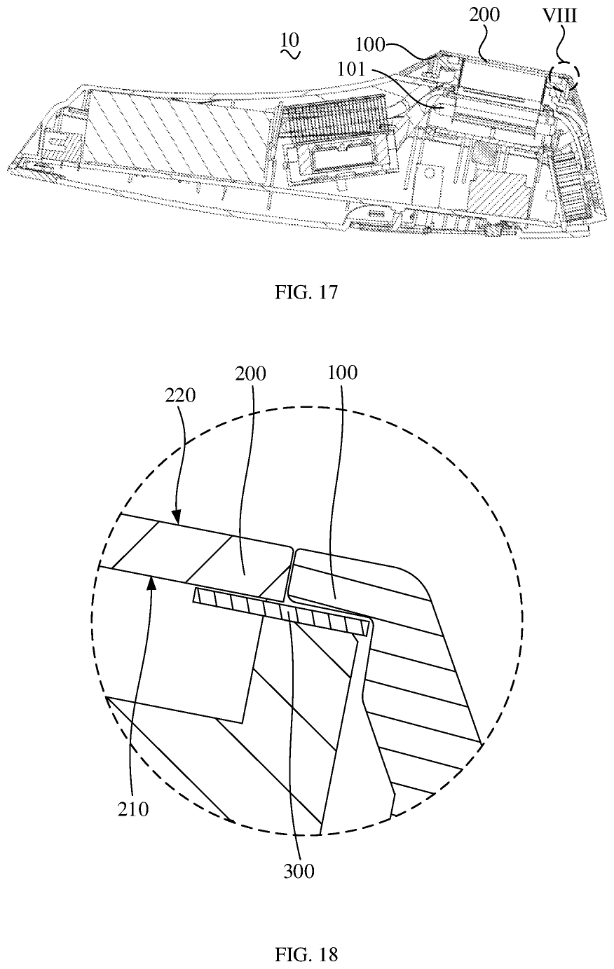

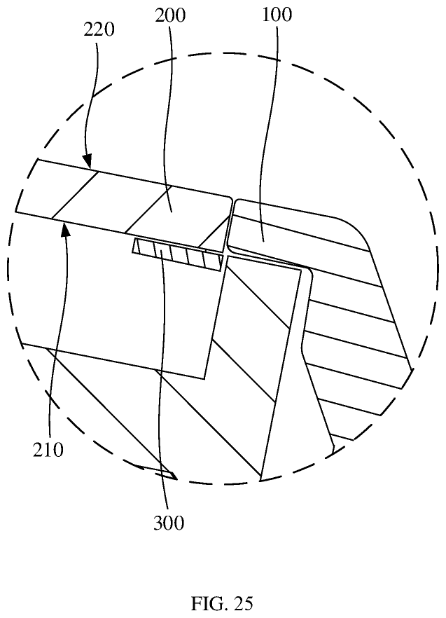

Referring to FIGS. 17 and 25, in some embodiments, a portion of the skin sensing component 300 is stacked with the sensing medium 200, and another portion of the skin sensing component 300 is stacked with the housing 100. For example, when the skin of the user comes into contact with the sensing medium 200, the sensing medium 200 serves as an insulating medium. The skin sensing component 300, the sensing medium 200, and the skin of the user may cooperatively form an identification capacitor, thereby resulting in a change in the total capacitance value of the capacitance circuits of the skin sensing component 300. As such, the skin treatment device 10 determines that the sensing medium 200 is in contact with the user of the skin through the skin sensing component 300, thereby controlling the light source assembly 101 to emit light toward the sensing medium 200. For example, when the skin of the user comes into contact with the housing 100, the housing 100 serves as an insulating medium. The skin sensing component 300, the housing 100, and the skin of the user may cooperatively form an identification capacitor, thereby resulting in a change in the total capacitance value of the capacitance circuits of the skin sensing component 300. As such, the skin treatment device 10 determines that the housing 100 is in contact with the user of the skin through the skin sensing component 300, thereby controlling the light source assembly 101 to emit light toward the sensing medium 200. Since the skin sensing component 300 is stacked with the sensing medium 200 and the housing 100, the skin treatment device 10 can sense the skin of the user in a wider area, thereby improving the overall sensing sensitivity of the skin treatment device 10.

In addition, the user can directly contact the housing 100 or the sensing medium 200 to trigger the light source assembly 101 to emit light. Thus, the user does not need to pay special attention to the contact location of the skin during use, which reduces the operation burden of the user and improves the convenience of the skin treatment device 10 during the treatment process.

Moreover, the skin treatment device 10 achieves dual detection of the contact with the sensing medium 200 and the housing 100 through a single skin sensing component 300, which helps to increase the area of the skin capable of being sensed by the skin sensing component 300 of the skin treatment device 10. Thus, the skin treatment device 10 can sense the skin more accurately, thereby reducing the risk of light leakage in the skin treatment device 10.

It can be understood that the skin sensing component 300 may be in direct contact with the housing 100, for example, the skin sensing component 300 may be directly adhered to the housing 100. Alternatively, the skin sensing component 300 may also be in indirect contact with the housing 100, for example, the skin sensing component 300 may be in contact with the housing 100 through a conductor.

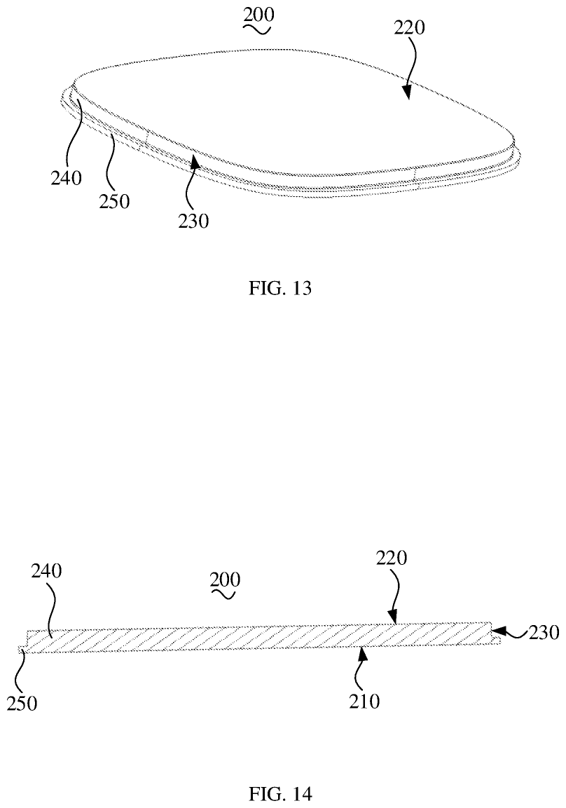

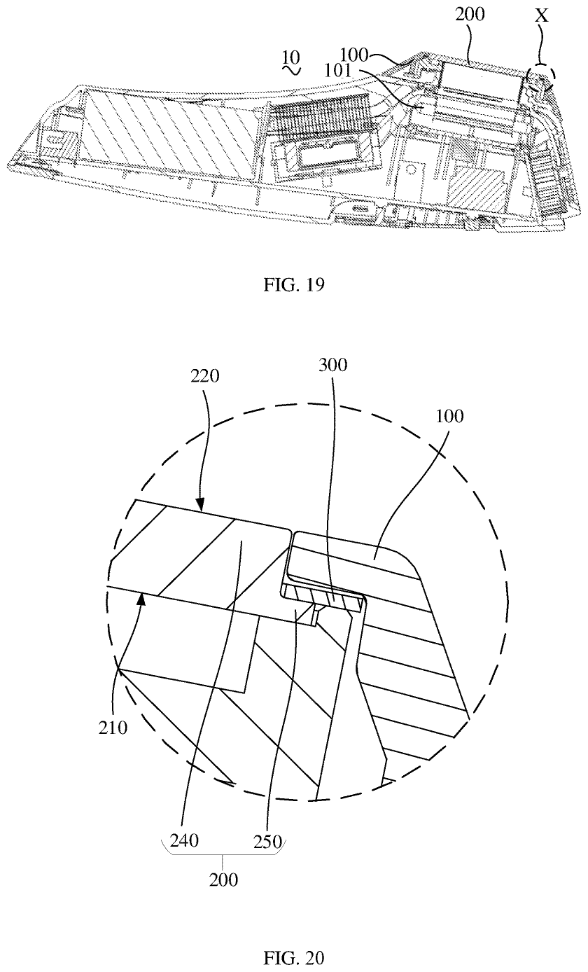

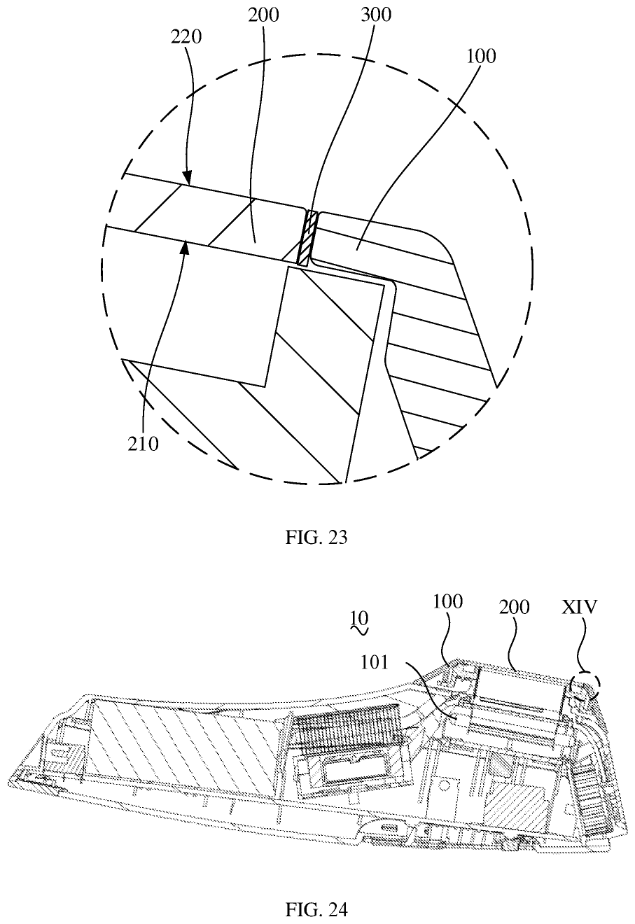

Referring to FIGS. 13 to 25, in some embodiments, the sensing medium 200 may be sapphire, and the sensing medium 200 may be sheet-shaped. The sensing medium 200 may include a light incident surface 210, a light output surface 220, and a connecting side surface 230. The light output surface 220 may be used to be in contact with the skin. The light emitted by the light source assembly 101 can pass through the light incident surface 210 and the light output surface 220 in sequence. The connecting side surface 230 may be connected between the light incident surface 210 and the light output surface 220. The skin sensing component 300 is at least partially attached to the connecting side surface 230 and/or the light incident surface 210 (such as being attached to the contact area 212 of the light incident surface 210). As such, the skin sensing component 300 is not in direct contact with the skin of the user but being attached to the connecting side surface 230 and/or the light incident surface 210 of the sensing medium 200, thereby avoiding direct electrical contact between the skin sensing component 300 and the human body, reducing the risk of electric shock accidents when the user uses the skin treatment device 10, and improving the electrical safety performance of the skin treatment device 10 during use. When the skin treatment device 10 includes the refrigeration component 400 mentioned above, the support bracket 600 may also be provided with another installation space to receive the skin sensing component 300. Therefore, the refrigeration component 400 and the skin sensing component 300 may be provided on the light incident surface 210 through the support bracket 600 and located at different portions of the contact area 212.

In addition, the skin sensing component 300 is not directly exposed from the light output surface 220. Thus, frequent friction between the skin sensing component 300 and the skin of the user may be avoided, which helps to reduce the wear and contamination of the skin sensing component 300 and extend the service life. Furthermore, the sensitivity and accuracy of the skin sensing component 300 is maintained, such that the skin sensing component 300 can work stably.

Moreover, since the skin sensing component 300 is not in direct contact with the skin, the accumulation of dirt and oil on the skin sensing component 300 is reduced, which helps users to clean and maintain the skin treatment device 10.

In some embodiments, the housing 100 may include a cavity 110 for receiving the light source assembly 101 and/or the sensing medium 200. The housing 100 may have a sidewall 120 that forms the cavity 110. The overall contour of the skin sensing component 300 may be substantially flat. The skin sensing component 300 may have a first sensing side 310 and a second sensing side 320 opposite to each other along the thickness direction.

In some embodiments, as shown in FIG. 25, the first sensing side 310 may face the light source assembly 101, and the second sensing side 320 may be in contact with the light incident surface 210 (such as in the contact area 212 of the light incident surface 210). When the skin comes into contact with the sensing medium 200, the skin serves as a pole plate of a capacitor, the sensing medium 200 serves as an insulating medium of the capacitor, and the second sensing side 320 serves as another pole plate of the capacitor. The skin, the sensing medium 200, and the second sensing side 320 cooperatively form an identification capacitor, resulting in a change in total capacitance value of the capacitance circuits of the skin sensing component 300. Thus, the skin treatment device 10 determines that the skin of the user is in contact with the sensing medium 200 through the skin sensing component 300, such that the skin treatment device 10 may control the light source assembly 101 to emit light.

In some implementations, as shown in FIG. 18, the first sensing side 310 may face the light source assembly 101, and the second sensing side 320 may be in contact with the light incident surface 210 (such as the contact area 212 of the light incident surface 210) and the sidewall 120. When the skin comes into contact with the sensing medium 200 and/or the housing 100, the sensing medium 200 and/or the housing 100 may serve as an insulating medium of the capacitor, the skin serves as a pole plate of the capacitor, and the second sensing side 320 serves as another pole plate of the capacitor plate, thereby forming an identification capacitor, resulting in a change in total capacitance value of the capacitance circuits of the skin sensing component 300. Thus, the skin treatment device 10 determines that the skin of the user is in contact with the sensing medium 200 through the skin sensing component 300, such that the skin treatment device 10 may control the light source assembly 101 to emit light.

In some embodiments, as shown in FIGS. 20, 21, and 23, the first sensing side 310 may be in contact with the sidewall 120, and the second sensing side 320 may be in contact with the connecting side surface 230. When the skin of the user comes into contact with the housing 100, the skin, the housing 100, and first sensing side 310 cooperatively form an identification capacitor. And/or, when the skin of the user comes into contact with the sensing medium 200, the skin, the housing 100, and the second sensing side 320 cooperatively form an identification capacitor, resulting in a change in total capacitance value of the capacitance circuits of the skin sensing component 300. Thus, the skin treatment device 10 determines that the skin of the user is in contact with the sensing medium 200 and/or the housing 100 through the skin sensing component 300, such that the skin treatment device 10 may control the light source assembly 101 to emit light.

Referring to FIGS. 13, 14, 20, and 21, in some embodiments, the sensing medium 200 may include a light-transmitting body 240 and an installation boss 250 connected to each other. The installation boss 250 protrudes relative to the light-transmitting body 240 towards the connecting side surface 230. The skin sensing component 300 may be disposed between the housing 100 and the light-transmitting body 240. Alternatively, the skin sensing component 300 may also be positioned between the housing 100 and the installation boss 250. As such, the sensing medium 200 and the housing 100 can clamp and fix the skin sensing component 300, such that the skin sensing component 300 is stably installed between the sensing medium 200 and the housing 100, thereby enabling the skin sensing component 300 to work normally. In addition, the first sensing side 310 is in direct contact with the sidewall 120, and the second sensing side 320 is in direct contact with the connecting surface side surface 230.

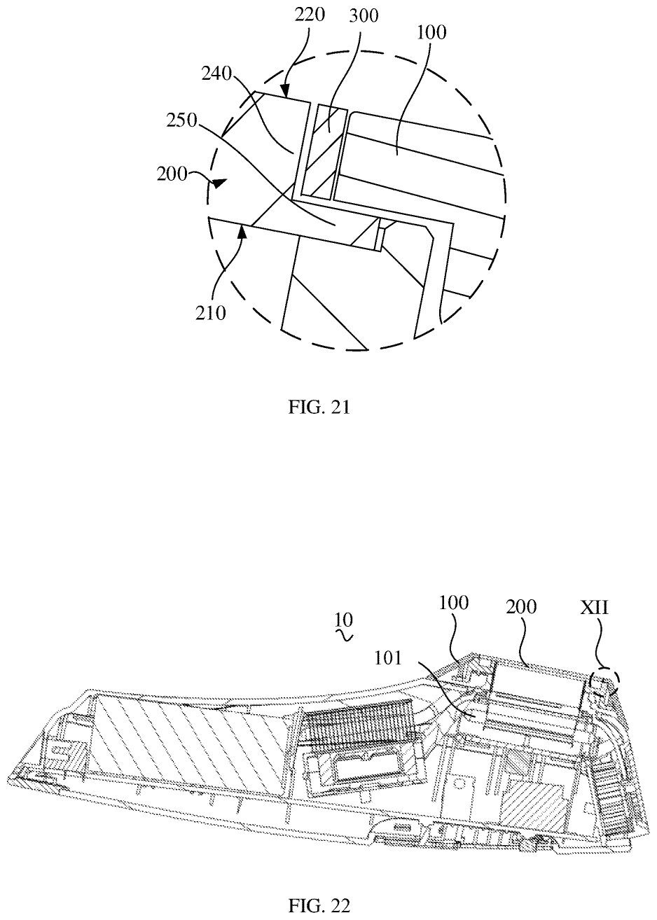

In some embodiments, as shown in FIGS. 20 and 21, the second sensing side 320 is in contact with the connecting side surface 230 of the light-transmitting body 240 or the connecting side surface 230 of the installation boss 250. Since the skin sensing component 300 is in contact with the connecting side surface 230 of the light-transmitting body 240 or the connecting side surface 230 of the installation boss 250, the skin sensing component 300 is prevented from blocking the light incident surface 210, thereby reducing the risk of the skin sensing component 300 blocking the output light of the light source assembly 101.

Referring to FIGS. 24 and 25, in some embodiments, the skin sensing component 300 may be entirely located on the sensing medium 200 and in contact with the light incident surface 210. Only when the skin is in direct contact with the sensing medium 200 can the skin treatment device 10 determine that the skin is in contact with the sensing medium 200 through the skin sensing component 300. As such, the light source assembly 101 is triggered to work, thereby improving the targeted and accurate treatment of the skin treatment device 10, and effectively utilizing the light energy of the light source assembly 101 to reduce resource waste.

In some embodiments, the light output surface 220 of the sensing medium 200 may be flush with the surface of the housing 100, such that the surface of the skin treatment device 10 is flat, which allows the skin of the user to be quickly and fully attached to the sensing medium 200.

In some embodiments, the light output surface 220 of the sensing medium 200 protrudes from the surface of the housing 100. That is, the light output surface 220 of the sensing medium 200 exceeds and is higher than the surface of the housing 100, such that the skin of the user can preferentially come into contact with the sensing medium 200. In addition, the protruding sensing medium 200 serves as a natural guiding device, thereby allowing the user to quickly bring the skin into contact with the sensing medium 200.

In some embodiments, the skin sensing component 300 may be a flexible circuit board. The flexible circuit board is composed of an insulating substrate with high flexibility (such as polyimide film) and conductive paths formed thereon. The flexible circuit board may be designed in any shape to adapt to the complex structure inside the device. Specifically, the flexible circuit board is precisely cut and shaped to fit along specific areas such as the light incident surface 210 and the connection side surface 230 of the sensing medium 200 of the skin treatment device 10, and even the sidewall 120 of the housing 100, thereby ensuring precise sensing even on curved surfaces or in a narrow space of the skin treatment device 10.

In addition, the flexible circuit board has good bending resistance, such that the skin sensing component 300 can be better attached to and installed onto the housing 100 and/or the sensing medium 200. Moreover, the lightweight and thin characteristics of the flexible circuit board reduces the overall weight and volume of the skin treatment device 10, such that the user may feel more lightweight and comfortable when holding or operating the skin treatment device 10.

In some embodiments, the skin sensing component 300 may be a metal-plated sensing component. The metal-plated sensing component may include a first metal plating layer, an insulating medium, and a second metal plating layer. The insulating medium may be located between the first metal plating layer and the second metal plating layer, such that the metal-plated sensing component serves as a reference capacitor. The first metal plating layer and the second metal plating layer may be thin and uniform metal films (such as copper, silver, gold, etc.) formed on key contact surfaces of the sensing medium 200, the housing 100, or the internal structures of the device through by processes such as physical vapor deposition (PVD), chemical vapor deposition (CVD), or electroplating.

In addition, the metal-plated sensing component has strong wear and oxidation resistance, and can maintain good sensing performance even under long-term use, thereby extending the service life and maintenance cycle of the skin treatment device 10.

Moreover, the first metal plating layer or the second metal plating layer may be directly deposited on key portions of the device without the need for additional installation components, thereby simplifying the internal structures of the skin treatment device 10, reducing the assembly steps of the skin treatment device 10, and reducing the device volume, and improving the compactness and aesthetics of the skin treatment device 10.

In some embodiments, the skin sensing component 300 may be annular. The annular skin sensing component 300 can provide a sensing range of 360 degrees, thereby allowing the skin of the user to be quickly sensed from any direction or angle when the skin is in contact with the skin treatment device 10, and improving the convenience and performance of the skin treatment device 10 during use. In addition, since the skin sensing component 300 is annular, the skin treatment device 10 will not have very strict requirements on the contact position of the skin of the user, such that the user may feel free to use the skin treatment device 10 without deliberately searching for a specific angle of contact position.

In some embodiments, there are multiple skin sensing components 300. The skin sensing components 300 may be arranged at intervals along a periphery of the sensing medium 200. Since the skin sensing components 300 are arranged at intervals along the periphery of the sensing medium 200, even when one or more skin sensing components 300 are damaged or fail due to some reasons, the remaining skin sensing components 300 may still work normally, such that the skin treatment device 10 may continue to accurately sense the contact of the skin, thereby reducing the risk of overall functional failure of the skin treatment device 10 due to a single fault, and thus improving the stability and reliability of the skin treatment device 10.

In some embodiments, the light source assembly 101 may include an LED tube. Alternatively, the light source assembly 101 may also include an IPL lamp, which may be selected according to the actual situations.

Clause 1: A skin treatment device comprising:
a housing equipped with a light source assembly therein;
a sensing medium made of crystalline material, and the sensing medium partially exposed from a surface of the housing and configured to allow light generated by the light source assembly to exit; and
a skin sensing component at least partially stacked with the sensing medium.

Clause 2: The skin treatment device of Clause 1, wherein a portion of the skin sensing component is stacked with the light-transmitting component, and another portion of the skin sensing component is stacked with the housing.

Clause 3: The skin treatment device of Clause 1, wherein the sensing medium comprises a light incident surface, a light output surface, and a connection side surface, the connection side surface is connected between the light incident surface and the light output surface, and the skin sensing component is at least partially in contact with the connection side surface and/or the light incident surface.

Clause 4: The skin treatment device of Clause 3, wherein the housing defines a cavity for receiving the light source assembly and/or the sensing medium, the housing comprises a sidewall for forming the cavity, and the skin sensing component has a first sensing side and a second sensing side opposite to each other along the thickness direction of the skin sensing component;
the first sensing side faces the light source assembly, the second sensing side is in contact with the light incident surface; or, the first sensing side is in contact with the sidewall, and the second sensing side is in contact with the connecting side.

Clause 5: The skin treatment device of Clause 4, wherein the sensing medium comprises a light-transmitting body and an installation boss connected to each other, the installation boss protrudes relative to the light-transmitting body toward the connection side surface, and the skin sensing component is located between the housing and the light-transmitting body; or, the skin sensing component is located between the housing and the installation boss.

Clause 6: The skin treatment device of Clause 5, wherein the second sensing side is in contact with the connecting side surface at the translucent body or the connecting side surface at the installation boss.

Clause 7: The skin treatment device of Clause 3, wherein the skin sensing component is entirely located on the sensing medium and in contact with the light incident surface.

Clause 8: The skin treatment device of any one of Clauses 1 to 7, wherein the light output surface of the sensing medium is flush a surface of the housing; or, the light output surface of the sensing medium protrudes from the surface of the housing.

Clause 9: The skin treatment device of any one of Clauses 1 to 7, wherein the skin sensing component is a flexible circuit board or a metal-plated sensing component.

Clause 10: The skin treatment device of any one of Clauses 1 to 7, wherein the skin sensing component is annular.

Clause 11: The skin treatment device of any one of Clauses 1 to 7, wherein the number of the skin sensing component(s) is multiple, and the multiple skin sensing components are arranged at intervals along a periphery of the sensing medium.

Furthermore, it can be understood that the above embodiments are illustrative of the present application. The technical solutions of embodiments can be arbitrarily combined when the technical features or the structures do not conflict with each other and when the purpose of the present application is not violated.

In the several embodiments provided in the present application, it should be understood that the disclosed system, apparatus, and method can be implemented in other ways. For example, the device embodiments described above are only illustrative. For example, the division of units is only based on logical functions. In practical implementation, there may be other division methods, for example, multiple units or components may be combined or integrated into another system, or for example, some features may be ignored or not executed. On the other hand, the mutual coupling, direct coupling, or communication connection displayed or discussed above can be indirect coupling or communication connection through some interfaces, devices or units, which can be electrical, mechanical or other forms of connection.

The units described as separate components may or may not be physically separated from each other. The components displayed as units may or may not be physical units, i.e., such units can be located in one place or distributed across multiple network units. Some or all of the units can be selected according to actual needs to achieve the purpose of the embodiments.

The above embodiments are only for describing but not intended to limit the present disclosure. Although the embodiments of the present disclosure have been described, those having ordinary skill in the art can understand that changes may be made within the principles of the present disclosure, up to and including the full extent established by the broad general meaning of the terms used in the claims. It will, therefore, be appreciated that the embodiments described above may be modified within the scope of the claims.

What is claimed is:

1. A skin treatment device (1) comprising:
a light source assembly (101);
a light-transmitting component (200), the light-transmitting component (200) comprising a light incident surface (210) and a light output surface (220), the light incident surface (210) facing the light source assembly (101), and the light incident surface (210) being configured to guide light generated by the light source assembly (101) into the light-transmitting component (200); and
at least one refrigeration component (400) located between the light-transmitting component (200) and the light source assembly (101), the refrigeration component (400) being in contact with the light incident surface (210), and at least a portion of the light incident surface (210) exposed from the at least one refrigeration component (400);
wherein the light incident surface (210) comprises a light incident area (211) and a contact area (212), the light incident area (211) is located at an interior area of the light incident surface (210), the contact area (212) is located at an edge area of the light incident surface (210), the light incident area (211) is configured to guide the light generated by the light source assembly (101) into the light-transmitting component (200), and the at least one refrigeration component (400) is in contact with the contact area (212);
wherein the skin treatment device (1) further comprises a support bracket (600), the support bracket (600) is located on a side of the light-transmitting component (200) facing the light source assembly (101), the support bracket (600) is connected to the light-transmitting component (200) and in contact with the contact area (212), the support bracket (600) defines at least one installation space (620), and the at least one refrigeration component (400) is received in the at least one installation space (620).

2. (canceled)

3. The skin treatment device (1) according to claim 1, wherein a distance from the light incident surface (210) to the light output surface (220) is 1 mm to 10 mm.

4. (canceled)

5. The skin treatment device (1) according to claim 1, wherein the support bracket (600) further defines a light incident hole (610), the at least one installation space (620) is adjacent to the light incident hole (610), and the at least one refrigeration component (400) is exposed from a surface of the support bracket (600) which is in contact with the contact area (212).

6. The skin treatment device (1) according to claim 5, wherein the at least one refrigeration component (400) comprises a plurality of refrigeration components (400), the at least one installation space (620) comprises a plurality of installation spaces (620), the refrigeration components (400) corresponds to the installation spaces (620) one-to-one, the plurality of installation spaces (620) are distributed at sidewalls of the support bracket (600), and the plurality of refrigeration components (400) surround the light incident hole (610) and are in contact with the contact area (212).

7. The skin treatment device (1) according to claim 1, wherein the skin treatment device (1) further comprises a wire space (630), the wire space (630) communicates with the at least one installation space (620), each of the at least one refrigeration component (400) further comprises a first wire (410) and a second wire (420), the first wire (410) and the second wire (420) are located at a same end of the refrigeration component (400), and each of the first wire (410) and the second wire (420) extends through the wire space (630).

8. The skin treatment device (1) according to claim 1, wherein the skin treatment device (1) further comprises an installation bracket (500) and a heat dissipation component (700), the installation bracket (500) is located at a side of the support bracket (600) facing the light source assembly (101), the light source assembly (101) is mounted on the installation bracket (500); the heat dissipation component (700) is located between the support bracket (600) and the installation bracket (500), and is connected to and thermally conductive with an end of each of the at least one refrigeration component (400) away from the light incident surface (210).

9. The skin treatment device (1) according to claim 8, wherein the skin treatment device (1) further comprises a conductive component (900), the conductive component (900) is located between the support bracket (600) and the heat dissipation component (700), the conductive component (900) is connected to and thermally conductive with the at least one refrigeration component (400) and the heat dissipation component (700), and/or, the conductive component (900) is semi-annular or annular.

10. The skin treatment device (1) according to claim 9, wherein the installation bracket (500) comprises a flange (504) protruding toward the support bracket (600), the flange (504) defines a through hole (508), the through hole (508) communicates with the light incident hole (610), and the skin treatment device (1) further comprises:
a light converging component (160) mounted in the light incident hole (610) and/or the through hole (508).

11. The skin treatment device (1) according to claim 1, wherein the skin treatment device (1) further comprises:
an auxiliary light emitting component (170) located between the light source assembly (101) and the light-transmitting component (200).

12. The skin treatment device (1) according to claim 1, wherein the skin treatment device (1) further comprises a thermal conductive connector (180), the thermal conductive connector (180) is located between the light-transmitting component (200) and the at least one refrigeration component (400), the thermal conductive connector (180) is in contact with the light incident surface (210), and is connected to and thermally conductive with the light-transmitting component (200) and the at least one refrigeration component (400).

13. The skin treatment device (10) according to claim 1, wherein the skin treatment device (10) further comprises:
a housing (100), the light source assembly (101) located in the housing (100), and the light-transmitting component (200) partially exposed from the housing (100) and configured to allow the light generated by the light source assembly (101) to exit; and
at least one skin sensing component (300) each at least partially stacked with the light-transmitting component (200).

14. The skin treatment device (10) according to claim 13, wherein a portion of each of the at least one skin sensing component (300) is stacked with the light-transmitting component (200), and another portion of each of the at least one skin sensing component (300) is stacked with the housing (100).

15. The skin treatment device (10) according to claim 13 wherein the light-transmitting component (200) further comprises a connecting side surface (230), the connecting side surface (230) is connected between the light incident surface (210) and the light output surface (220), and each of the at least one skin sensing component (300) is at least partially in contact with the connecting side surface (230) and/or the light incident surface (210).

16. The skin treatment device (10) according to claim 15, wherein the housing (100) defines a cavity (110) for receiving the light source assembly (101) and/or the light-transmitting component (200), the housing (100) comprises a sidewall (120) for forming the cavity (110), and each of the at least one skin sensing component (300) comprises a first sensing side (310) and a second sensing side (320) opposite to each other along a thickness direction of the skin sensing component (300);
the first sensing side (310) faces the light source assembly (101), the second sensing side (320) is in contact with the light incident surface (210); or, the first sensing side (310) is in contact with the sidewall (120), and the second sensing side (320) is in contact with the connecting side surface (230).

17. The skin treatment device (10) according to claim 16, wherein the light-transmitting component (200) comprises a light-transmitting body (240) and an installation boss (250) connected to each other, the installation boss (250) protrudes relative to the light-transmitting body (240) toward the connecting side surface (230), the at least one skin sensing component (300) is located between the housing (100) and the light-transmitting body (240), or the at least one skin sensing component (300) is located between the housing (100) and the installation boss (250).

18. The skin treatment device (10) according to claim 17, wherein the second sensing side (320) is in contact with the connecting side surface (230) at the light-transmitting body (240) or the connecting side surface (230) at the installation boss (250).

19. The skin treatment device (10) according to claim 15, wherein each of the at least one skin sensing component (300) is entirely located on the light-transmitting component (200) and in contact with the light incident surface (210).

20. The skin treatment device (10) according to claim 13, wherein the light output surface (220) of the light-transmitting component (200) is flush with a surface of the housing (100), or the light output surface (220) of the light-transmitting component (200) protrudes from the surface of the housing (100).

21. The skin treatment device (10) according to claim 13, wherein each of the at least one skin sensing component (300) is a flexible circuit board or a metal-plated sensing component, and each of the at least one skin sensing component (300) is annular.

22. (canceled)

23. The skin treatment device (10) according to claim 13, wherein the at least one skin sensing components (300) comprises a plurality of skin sensing components (300), and the plurality of skin sensing components (300) are spaced from each other along a periphery of the light-transmitting component (200).
